(19) United States
(12) Patent Application Publication
(10) Pub. No.: US 2022/0105234 A1
(43) Pub. Date: Apr. 7, 2022

(54) ADHESIVE FOR OSSEOINTEGRATED PERCUTANEOUS DEVICES

(71) Applicant: Regents of the University of Minnesota, Minneapolis, MN (US)

(72) Inventors: Conrado Aparicio, Minneapolis, MN (US); Isha Mutreja, Woodbury, MN (US)

(21) Appl. No.: 17/495,648

(22) Filed: Oct. 6, 2021

Related U.S. Application Data
(60) Provisional application No. 63/088,620, filed on Oct. 7, 2020.

(51) Int. Cl. A61L 24/00; A61K 38/08; A61K 38/16; A61K 38/17; A61K 38/10; A61L 27/06

(52) U.S. Cl. CPC A61L 24/0089; A61K 38/08; A61K 38/164; A61K 38/1703; A61K 38/10; A61L 2300/404; A61L 24/0031; A61L 27/06; A61L 2400/12; A61L 2300/252; A61L 24/0036

(57) ABSTRACT

A bioactive adhesive for use in securing soft tissue to osseointegrated percutaneous devices includes a hydrogel precursor and a multiplicity of metal-containing mesoporous silicate nanoparticles dispersed throughout the hydrogel precursor. An antimicrobial peptide is adsorbed on surfaces of the mesoporous silicate nanoparticles, incorporated in the mesoporous silicate nanoparticles, or both. The metal-containing mesoporous silicate nanoparticles can include calcium, strontium or both and are configured to release the antimicrobial peptide over time. Adhering tissue to a metal surface includes disposing the bioactive adhesive on a metal surface, contacting a portion of tissue with the adhesive composition, and curing the adhesive composition, thereby adhering the portion of tissue to the metal surface.

### CROSS-REFERENCE TO RELATED APPLICATION

This application claims the benefit of U.S. Patent Application Ser. No. 63/088,620, filed Oct. 7, 2020, which is incorporated by reference herein in its entirety.

### TECHNICAL FIELD

This invention relates to an adhesive for securing osseointegrated percutaneous devices to soft tissue.

### BACKGROUND

Osseointegration is a method of attaching a percutaneous device (e.g., an orthopedic limb prosthesis, a dental implant, or a bone-anchored hearing aid) directly to bone. An osseointegrated device typically includes a fixture that is implanted into bone and an abutment that transdermally connects the external prosthesis to the fixture. The fixture includes a biocompatible metal (e.g., a titanium alloy) and is typically microporous. The abutment has a polished surface to minimize contact at the skin penetration interface and can be combined with an antimicrobial coating or other types of surface antimicrobial treatments to prevent bacterial adhesion.

### SUMMARY

This disclosure describes an adhesive for securing soft tissue (e.g., skin or mucosa) to osseointegrated percutaneous devices (e.g., orthopedic limb prostheses, dental implants, and bone-anchored hearing aids). The adhesive, formulated to support re-epithelialization, prevent infection, and promote healing, includes an antimicrobial peptide releasably stored in mesoporous silicate nanoparticles (MSNs). The antimicrobial peptide prevents bacterial colonization without interfering with migration, attachment and proliferation of epidermal keratinocytes and fibroblasts from the surrounding skin into the adhesive. The adhesive is applied as a viscous liquid and cured to form a crosslinked hydrogel that adheres soft tissue (e.g., a skin flap) to a metal surface of the percutaneous device (e.g., during surgery), creating a tight seal between the skin and the metal surface.

In a first general aspect, a bioactive adhesive composition includes a hydrogel precursor and a multiplicity of metal-containing mesoporous silicate nanoparticles dispersed throughout the hydrogel precursor. An antimicrobial peptide is adsorbed on surfaces of the mesoporous silicate nanoparticles, incorporated in the mesoporous silicate nanoparticles, or both.

Implementations of the first general aspect can include one or more of the following features.

In some implementations, the composition is photopolymerizable (e.g., under visible light). The hydrogel precursor can include gelatin methacryloyl (GelMA). In some cases, the metal-containing mesoporous silicate nanoparticles can include calcium or calcium and strontium, and have a diameter in a range of about 150 nm to about 250 nm. In certain cases, the metal-containing mesoporous silicate nanoparticles include strontium and have a diameter of the nanoparticles is in a range of about 350 nm to about 450 nm. The composition can include 0.5 wt % to 50 wt % of the composition.

The antimicrobial peptide can have a loading efficiency of at least 50%. In some cases, the anti-microbial peptide includes GL13K, 1010, DJK2, DJKS, hlf1-11, nisin, LL-37 or a combination thereof. The metal-containing mesoporous silicate nanoparticles can be configured to release the antimicrobial peptide over time.

The composition can be configured to adhere to skin, metal, or both. The composition can be configured to promote the release of cytokines from soft tissue in contact with the composition.

In a second general aspect, adhering skin to a metal surface includes disposing the adhesive composition of the first general aspect on a metal surface, contacting a portion of the skin with the adhesive composition, and curing the adhesive composition, thereby adhering the portion of the skin to the metal surface.

Implementations of the second general aspect can include one or more of the following features.

In some cases, curing the adhesive composition can include irradiating the adhesive composition with visible light. In certain cases, curing the adhesive composition includes converting the hydrogel precursor to a hydrogel. The metal surface can be a surface of a percutaneous implant. In one example, the metal surface includes titanium. Adhering the portion of skin to the metal surface typically forms a seal between the portion of the skin and the metal surface.

The bioactive adhesive composition has various advantages. The nanoparticles prolong the storage and release of the antimicrobial peptide, thereby reducing mono-and multi-species biofilm in contact with the adhesive and promoting adhesion between the metal implant and skin. In addition, release of metals as the nanoparticles disintegrate supports the release of growth factors from fibroblasts, thereby promoting re-epithelialization and healing.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts the use of a bioactive gelatin-based hydrogel charged with antimicrobial peptide (AMP)-loaded nanoparticles to promote soft tissue attachment to metal.

FIG. 2A shows a structural formula of the adhesive. FIG. 2B depicts a process for fabricating mesoporous silicate nanoparticles (MSNs) and loading the MSNs with drugs (e.g., AMP).

FIGS. 3A-3F show scanning electron microscopy (SEM) and transmission electron microscopy (TEM) micrographs of CaMSN, Sr-CaMSN, and SrMSN.

FIGS. 4A and 4B show bar graphs related to compatibility assessment of different concentrations of MSNs against human mesenchymal stromal cells (MSCs) as determined by alamarBlue™ assay.

FIG. 5 shows sustained release of 5-(and-6)-carboxytetramethylrhodamine (TAMRA)-labelled GL13K (AMP) from different MSNs in phosphate buffer saline at 37° C.

FIGS. 6A-6C show metabolic activity of MSCs cultured in presence of AMP-loaded MSNs.

FIGS. 7A-7C show metabolic activity of USA300 cultured in the presence of AMP-loaded MSNs, MSN alone, and AMP added directly to a bacterial suspension.

FIGS. 8A-8C show crystal violet biomass of USA300 cultured in the presence of AMP-loaded MSNs, MSN alone, and AMP, respectively, added directly to a bacterial suspension.

FIG. 9A shows the ability of the adhesive to hold two pieces of human skin tissue together post cross-linking as shown in the optical micrograph. FIG. 9B shows a schematic of the bonding in FIG. 9A. FIG. 9C is an image showing migration of cells from the skin tissue into the adhesive after 14 days of culture in media.

FIG. 10 depicts migration of encapsulated MSCs from gelatin methacryloid (GelMA) hydrogels of varying stiffness onto an underlying titanium substrate.

FIGS. 11A and 11B show mesoporous MSN-laden GelMA support cell viability (day 1) and proliferation (day 7), respectively, of encapsulated MSCs as determined by alamarBlue™

FIG. 12 shows metabolic activity of USA300 cultured on GelMA, GelMA with SrMSN, and GelMA with AMP-loaded SrMSN.

### DETAILED DESCRIPTION

This disclosure describes a biocompatible, bioactive adhesive for osseointegrated percutaneous devices that includes a biological scaffold combined with bioactive peptides that upregulate keratinocyte proliferation and hemidesmosome formation. The adhesive is a gelatin-based hydrogel that includes nanoparticles loaded with antimicrobial peptide (AMP). Suitable nanoparticles include mesoporous calcium and strontium silicate. The nanoparticles prolong carrying and release of AMP, thereby reducing mono-and multi-species biofilm in contact with the adhesive and promoting adhesion between the metal implant and skin. In addition, release of strontium and calcium as the nanoparticles disintegrate supports the release of cytokines such as basic fibroblast growth factor (bFGF) and vascular endothelial growth factor (VEGF) from fibroblasts, obviating the need for additional growth factors.

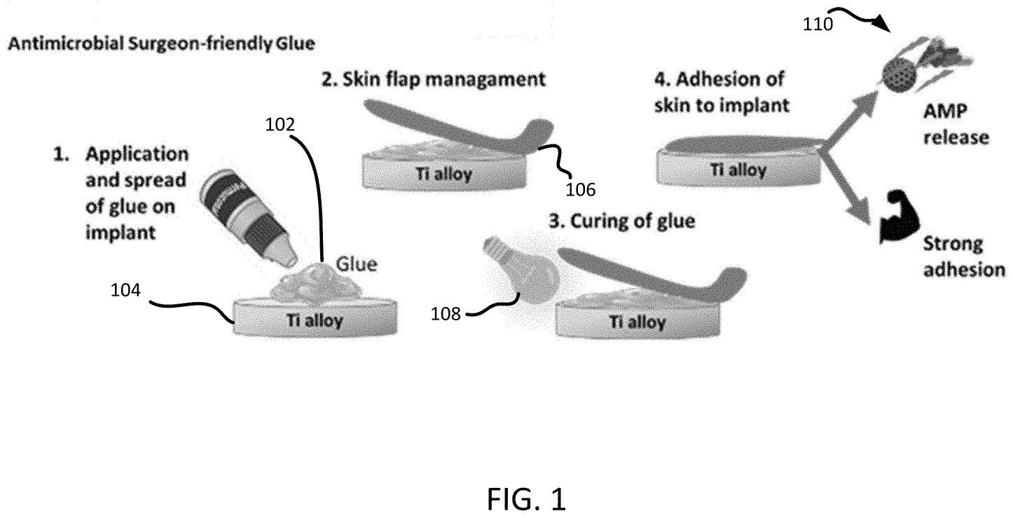

The adhesive can be applied during a surgical procedure. As depicted in FIG. 1, the adhesive 102 is applied to a surface of a metal implant 104 configured to contact skin. The skin 106 to be adhered to the surface of the metal implant 104 is positioned on the adhesive 102. The adhesive 102 is cured (e.g., with visible light 108), adhering the skin 106 to the implant 104, and AMP 110 is released from the cured adhesive.

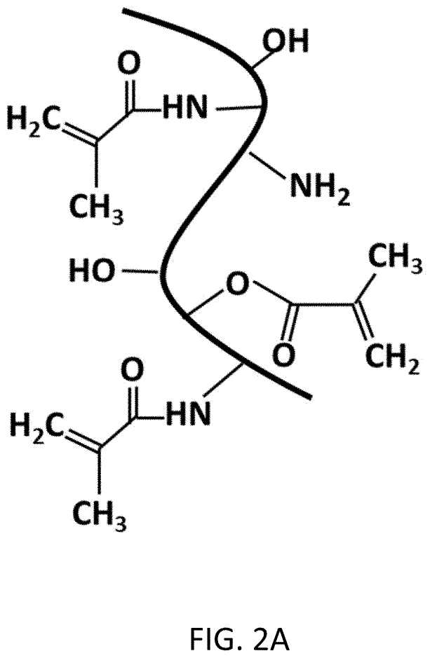

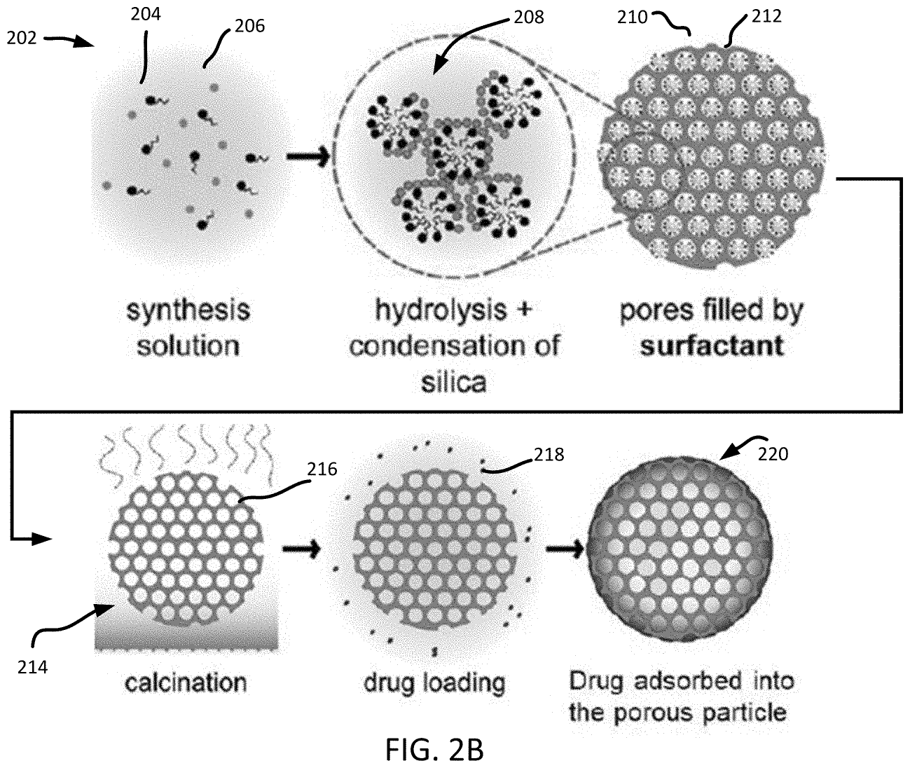

The adhesive described herein includes a photopolymerizable gelatin methacryloyl, depicted in FIG. 2A, combined with antimicrobial peptide (AMP) loaded metal mesoporous silicate nanoparticles (MSNs). The formulation allows loading small molecules, such as antimicrobial peptides, growth factors, and drug molecules, which are released at a slow rate over prolonged periods due to diffusion and biological degradation of MSNs. FIG. 2B depicts synthesis of metal MSNs. Metals used for the synthesis of MSNs can include calcium (CaMSNs), strontium (SrMSNs) or strontium-calcium (Sr-CaMSNs). The initial synthesis solution 202 contains a surfactant 204 and silica 206 together with the dissolved metal. After hydrolysis and condensation of the silica 208 to form porous silica nanoparticles 210 filled by the surfactant molecules 212, the nanoparticles are collected by centrifugation, washed, and lyophilized. The powdered nanoparticles are then calcinated 214 to remove surfactant by heating to form the MSN 216. The functional component 218 (e.g. AMP such as GL13K) is loaded into the MSN 216 by slow stirring at 37° C. to yield the loaded MSN 220.

The MSNs can be characterized for cytotoxicity against mesenchymal stem cells (MSCs) as well as chemically and structurally using scanning electron microscopy (SEM) with energy dispersive X-ray spectroscopy and wide-angle x-ray diffractometry. Peptide loading and release from MSNs can be assessed using the fluorescent-labelled GL13K AMP. CaMSNs and Sr-CaMSNs are typically 200±50 nm in diameter, and SrMSNs are typically 400±50 nm in diameter. The MSNs are amorphous and have a peptide-loading efficiency over 90% (e.g., 96±1%) with a slow release profile. SrMSNs, Sr-CaMSNs and CaMSNs typically release about 34% and about 11% of loaded AMP in about 24 h. The MSNs improve cell metabolic activity, thereby confirming cytocompatibility. The MSNs support cell growth over extended periods, thereby promoting bone regeneration with prolonged antibacterial capability when combined with AMP.

### EXAMPLES

### Materials Synthesis

Mesoporous Metal Silicates. CaMSN: 2.2 grams CTAB (cetyltrimethylammonium bromide, also known as hexadecyltrimethyl-ammonium bromide, h5882, Millipore Sigma) was added to 174 ml deionized (DI) water with 6 ml liquid NH3 and stirred for one hour. 10.4 grams Ca(NO₃)₂·4H₂O (Millipore Sigma, C1396) dissolved in 10 ml water along with 10 ml TEOS (Tetraethyl orthosilicate, Millipore Sigma, 131903) was added dropwise to CTAB solution and the reaction was continued for 4 hours. After 4 hours, nanoparticles were collected via centrifugation and were washed with DI water and lyophilized. The powder collected was calcinated at 550° C. for 6 hours. Sr-CaMSN: ((20% Sr, 80%Ca) 2.02 g of strontium nitrate (Millipore Sigma, 243426) and 8.32g of calcium nitrate) were synthesized using the same reaction conditions that were used for synthesizing CaMSN followed by centrifugation, lyophilization and calcination to collect the MSNs. SrMSN: (10.14g of strontium nitrate) were synthesized using the same reaction conditions that were used for synthesizing CaMSN followed by centrifugation, heat drying at 60° C., and calcination to collect the MSNs.

AMP-loaded MSNs. D-GL13K (Bachem) (peptide sequence GKIIKLKASLKLL with D-enantiomer amino acids) was chosen as a model antimicrobial peptide. 3 mg of D-GL13K was dissolved in sterile DI-water which was then used to solubilize 30 mg of MSNs. In order to ensure peptide loading within the pores of MSN, samples were left at 37° C. with slow stirring, after which samples were centrifuged and washed with DI-water. Other examples of AMPs that can be used include peptides 1018 (VRLIVAVRIWRR), DJK2 (D-enantiomers of VFWRRIRVWVIR), DJK5 (D-enantiomers of VQWRAIRVRVIR), hLF1-11 (GRRRRSVQWCA), LL-37 (LLGDFFRKSKEKIGKEFKRIVQRIKDFLRNLVPRTES), and the small protein nicin, or a combination thereof.

GelMA synthesis. GelMA was synthesized following a known protocol (https://doi.org/10.1038/nprot.2016.037), which is incorporated by reference herein. GelMA solution was prepared at 20 wt % in phosphate buffer saline, and hydrogels were fabricated at 10 wt % using 0.1 wt % LAP (lithium phenyl-2,4,6-trimethylbenzoylphosphinate, 900889-Millipore Sigma) as photo-initiator and 400 nm light source to initiate radical-based cross-linking.

GelMA-MSN nanocomposites. Different weight percentages of MSNs with or without AMP were mixed with the macromer, and the samples were cross-linked.

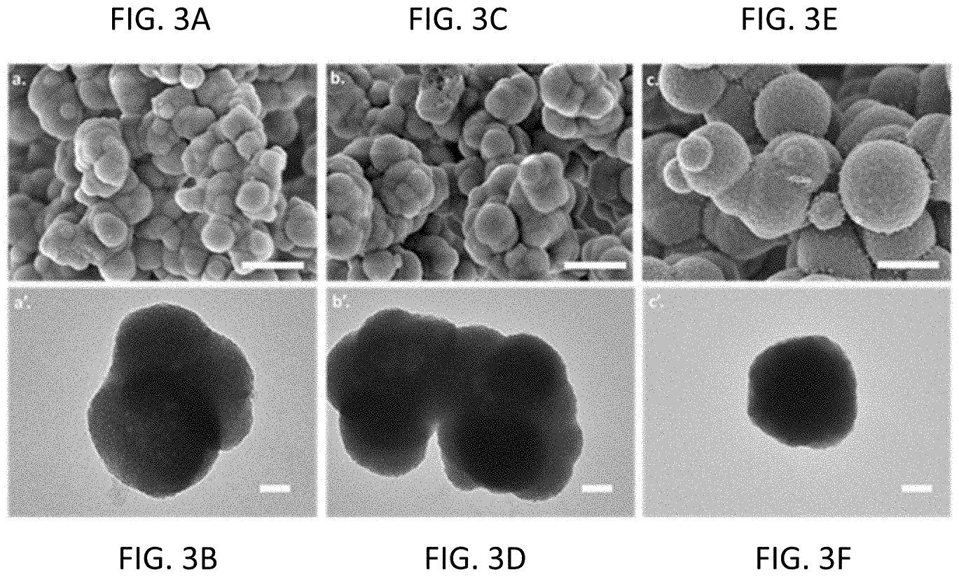

Substitution of Ca with Sr to form Sr-CaMSN or SrMSN. Samples were prepared by dissolving 1 mg of calcined CaMSN in DI-water which was then drop dried on carbon tape for imaging. For TEM, samples were drop-dried on carbon coated copper grids. Scanning electron microscopy was performed on a Hitachi SU8 electron microscope. FIGS. 3A-3F show SEM and TEM micrographs of CaMSN, Sr-CaMSN, and SrMSN Similar nanostructure morphology was observed for Sr-CaMSN and SrMSN. FIGS. 3A and 3B show SEM and TEM micrographs of CaMSN, respectively. FIGS. 3C and 3D show SEM and TEM micrographs of Sr-CaMSN, respectively. FIGS. 3E and 3F show SEM and TEM micrographs of SrMSN, respectively. The scale bars in FIGS. 3A, 3C, and 3E (SEM images) represent about 500 nm. The scale bars in FIGS. 3B, 3D, and 3F (TEM images) represent about 50 nm.

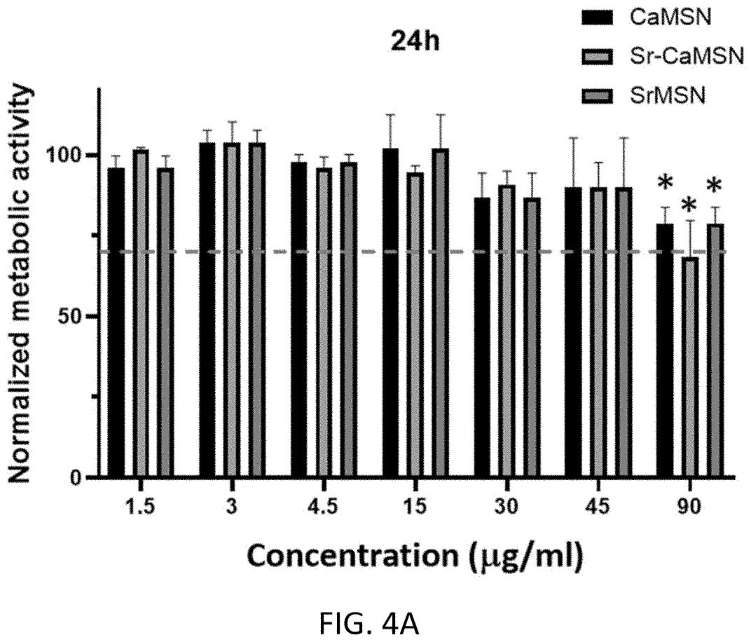

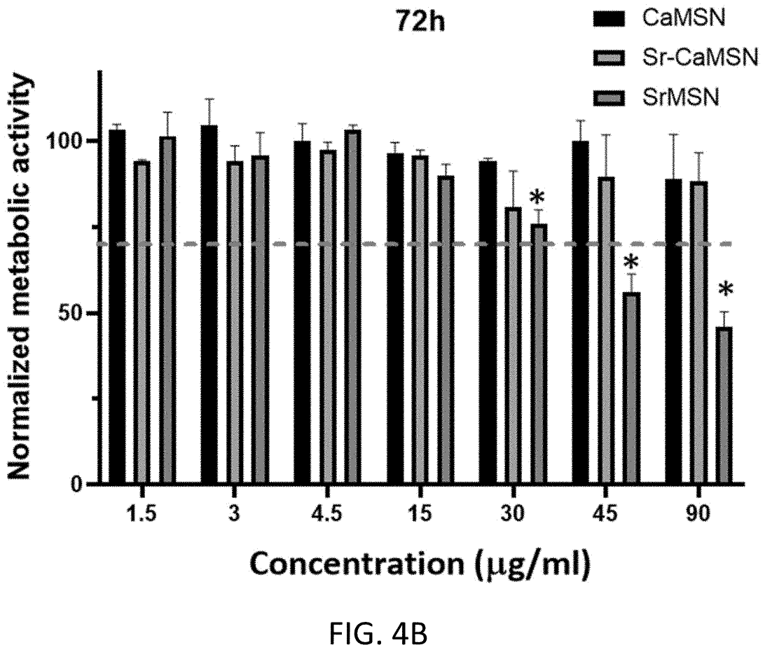

Assessing the cytotoxicity of as-prepared MSNs against human MSCs. Human MSCs were seeded in 48 well plates at a seeding density of 10000 cells/cm2 for 24 h, after which cells were treated with different concentrations of MSNs (1.5 μg/ml−90 μg/ml) for 24 h and 72 h. At each time point, metabolic activity was measured using alamarBlue™ (Thermo Fisher Scientific) following manufacturers protocol. Fluorescent intensity was read using a microplate reader (Synergy HT, Biotek) at 450 nm. No significant differences in fluorescent intensity (measure of metabolic activity) was determined for cells treated with lower concentrations (up to 45 μg/ml of different MSNs) after 24 h and 72 h. However, significant differences were observed in metabolic activity of MSCs treated with SrMSNs at concentrations higher than 30 μg/ml, whereas no MSN-mediated toxicity was observed for CaMSN and Sr-CaMSN. FIGS. 4A and 4B show bar graphs related to compatibility assessment of different concentrations of MSNs against MSCs as determined by alamarBlue™ assay. Statistical analysis: One-way ANOVA; Tukey post-hoc; letters with different symbol were significantly different (p<0.05).

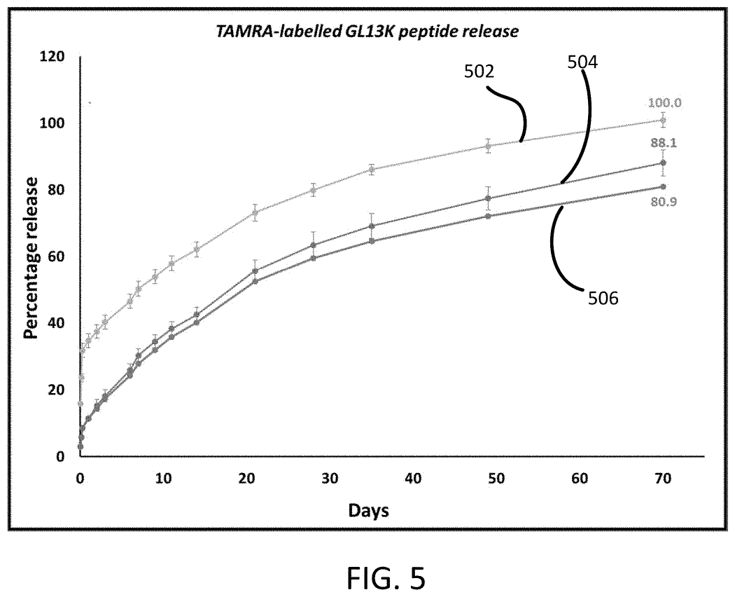

Loading and release profile of fluorescently-labelled GL13K under physiological conditions. TAMRA-labelled GL13K was dissolved in DI-water and loaded at a weight ratio of 1:10 (AMP:MSN) at 37° C. After 24 h, samples were centrifuged and washed with DI-water to remove unbound peptide. AMP-loaded MSNs were re-suspended in PBS and cumulative release was determined over prolonged periods. To determine the amount of peptide released, 500 μl of PBS was removed and 500 μl of fresh PBS was added. Burst release was observed in the first 24 h (˜35% of loaded AMP from SrMSN and 10% from CaMSN and Sr-CaMSN) followed by sustained release over the period of 70 days. FIG. 5 shows sustained release of TAMRA-labelled GL13K (AMP) from SrMSN 502, Sr-CaMSN 504, and CaMSN 506 in phosphate buffer saline at 37° C.

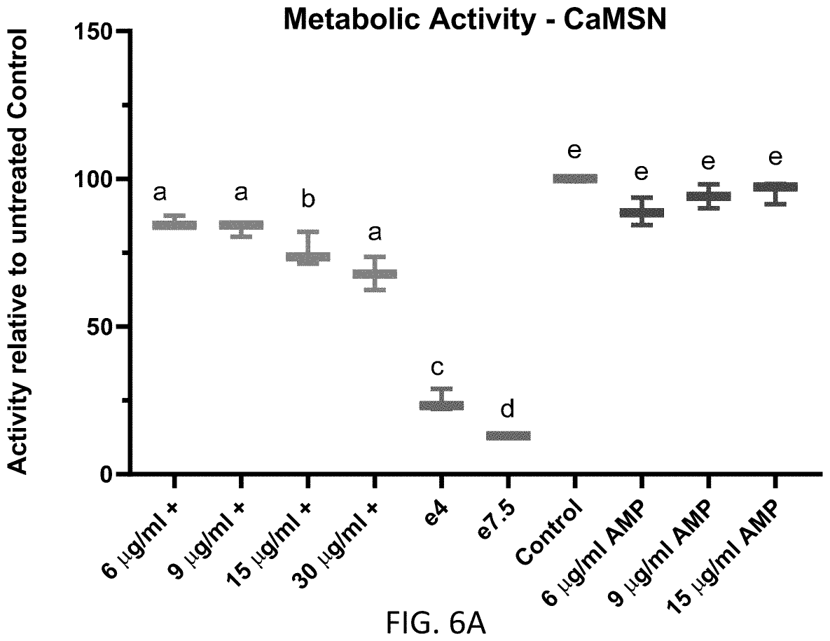

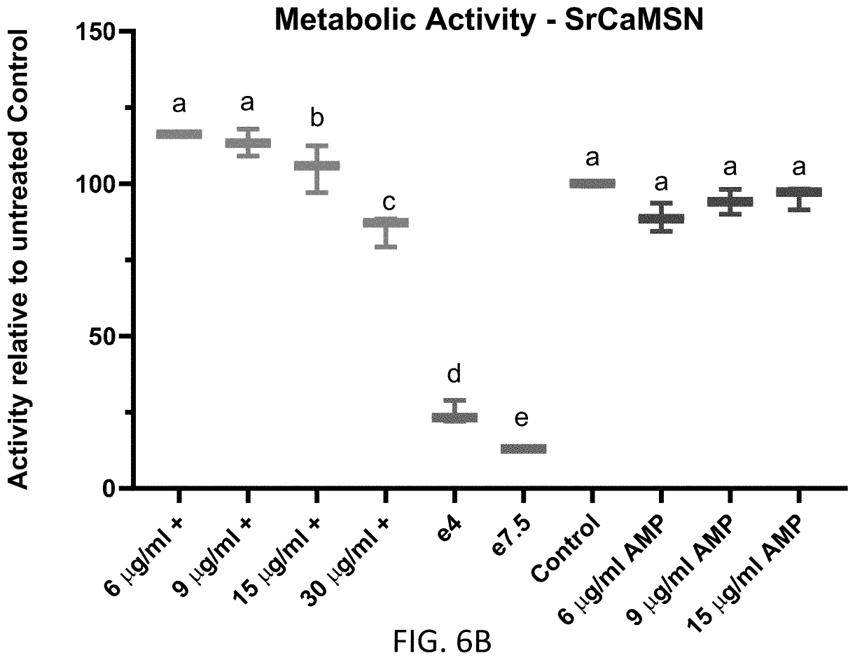

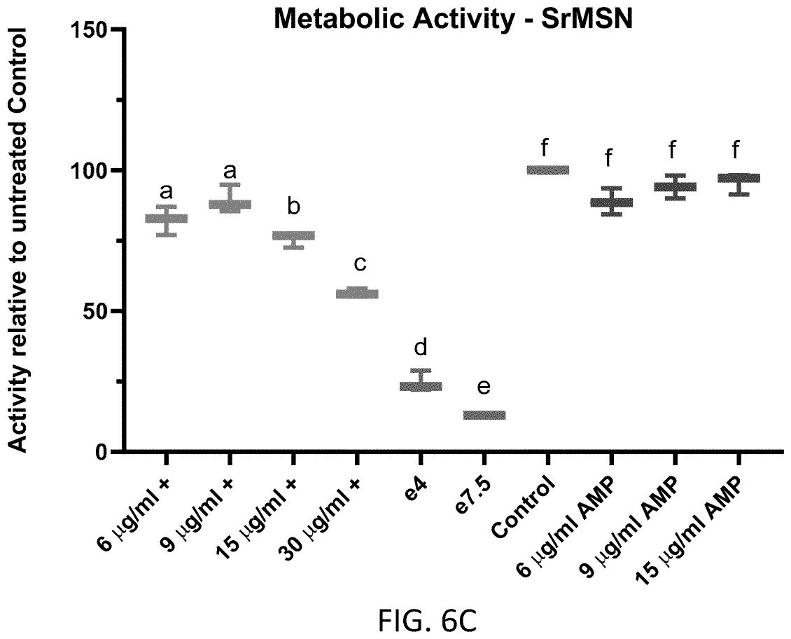

Assessing the cytotoxicity of GL13K-loaded MSNs against human MSCs following modified ISO 10993-5 standard. Human MSCs were seeded in 48 well plate at a seeding density of 10000 cells/cm' for 24h after which cells were treated with different concentrations of AMP-loaded MSNs (dispersed in PBS) at a concentration ranging from 6μg/ml −30 μg/ml for another 48h. Ethanol (4% and 7.5%) was used as positive control and PBS (vehicle control) as negative control. After 48 h, metabolic activity was measured using alamarBlue™ (Thermo Fisher Scientific) following manufacturers protocol. Fluorescent intensity was read using a microplate reader (Synergy HT, Biotek) at 450nm. Significant differences in fluorescent intensity (measure of metabolic activity) were determined for cells treated with AMP-loaded CaMSN and SrMSNs at different tested concentrations. However, no differences were observed for AMP-loaded Sr-CaMSN at lower concentrations. Results suggest that concentrations 15 μg/ml and higher showed dose dependent cytotoxicity for CaMSN and SrMSN whereas for AMP-Sr-CaMSN, cytotoxicity was only observed at 30 μg/ml. These results confirm dose-dependent cytotoxicity when cells were treated directly with the nanostructures. FIGS. 6A-6C show metabolic activity of MSCs cultured in presence of AMP-loaded MSNs. Statistical analysis: One-way ANOVA; post Bonferroni's test; letters with different symbol were significantly different (p<0.05).

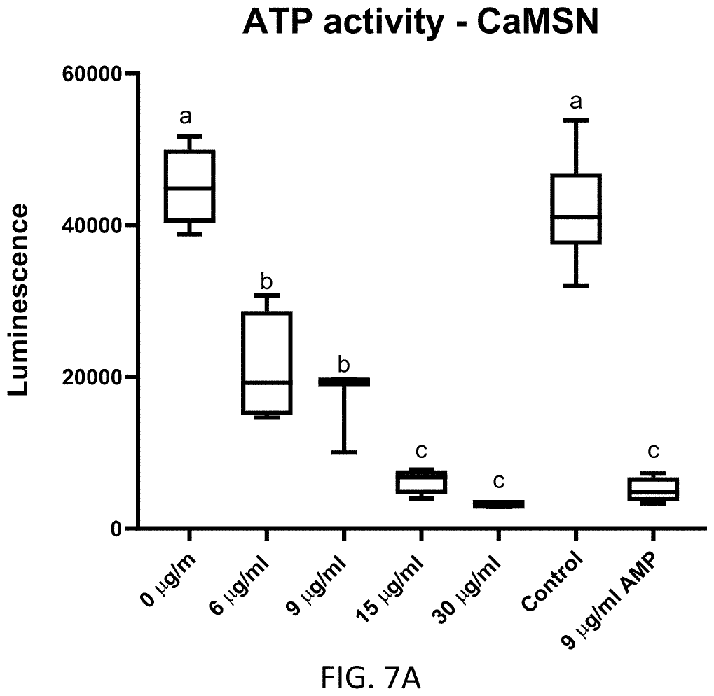

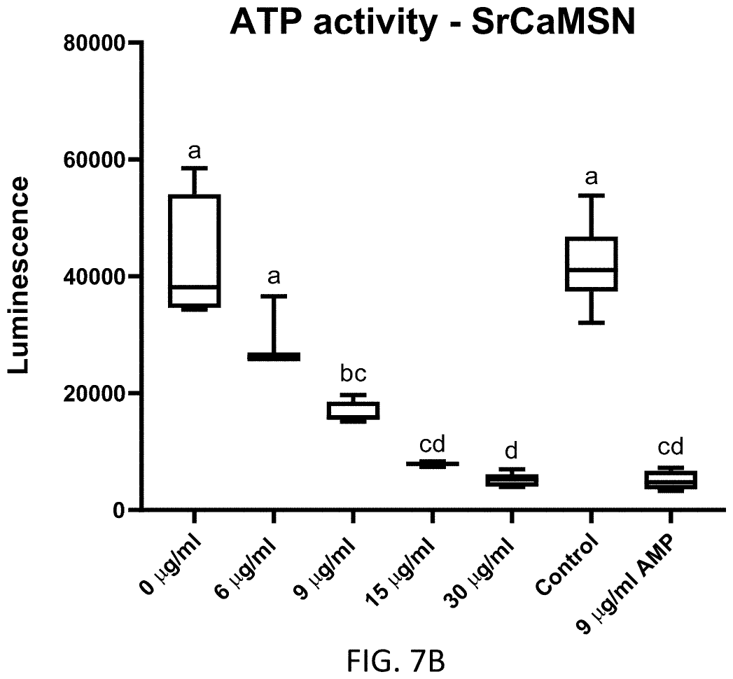

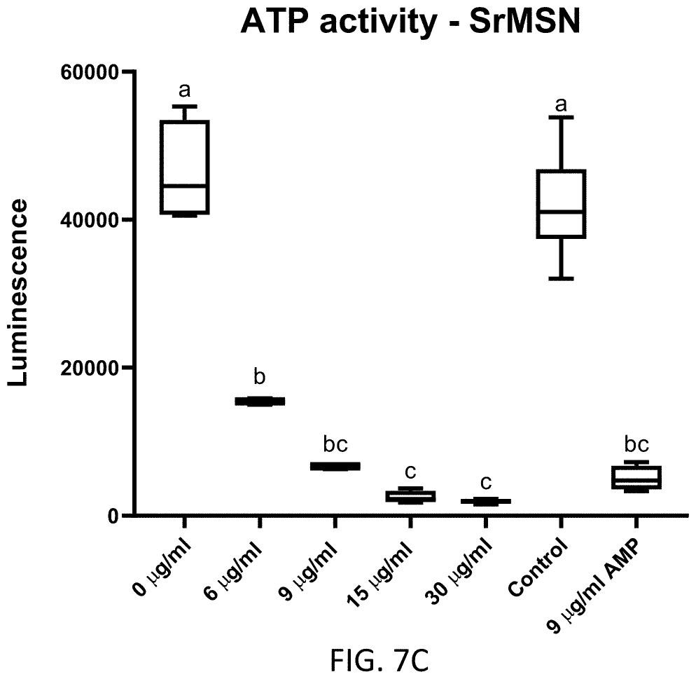

Assessing the antibacterial activity of GL13K-loaded MSNs against Staphylococcus aureus (USA300, MRSA strain). Overnight culture of S. aureus, USA300 prepared in tryptic soy broth (TSB, DF0370-17-3, Fisher Scientific) were used for assessing the antibacterial activity of AMP-loaded MSNs. USA300 is one of the primary causatives of implant associated infection and implant failure. The optical density of the overnight culture was adjusted to 0.05 and treated with different concentrations of AMP-loaded MSN and AMP alone and MSN (without AMP) and PBS (vehicle control) was used as positive and negative controls. The bacteria was cultured for 24 h under different conditions and metabolic activity tested using BacTiter-Glo™ microbial cell viability assay. The assay was performed using manufacturer's instruction and metabolic activity was measured by luminescence which was measured using microplate reader (Synergy HT, Biotek). Significant differences in luminescence intensity (measure of metabolic activity) was observed for bacteria treated with AMP alone or AMP-loaded MSNs relative to MSN alone and PBS control. The reduction in luminescence, that is bacteria viability, was observed in a dose dependent manner for cells treated with AMP-loaded CaMSN and SrMSNs at different tested concentrations. FIGS. 7A-7C show metabolic activity of USA300 cultured in presence of AMP-loaded MSNs, MSN alone and AMP added directly to the bacterial suspension. Values on x-axis correspond to the loaded AMP concentration. Statistical analysis: One-way ANOVA; post Bonferroni's test; letters with different symbol were significantly different (p<0.05).

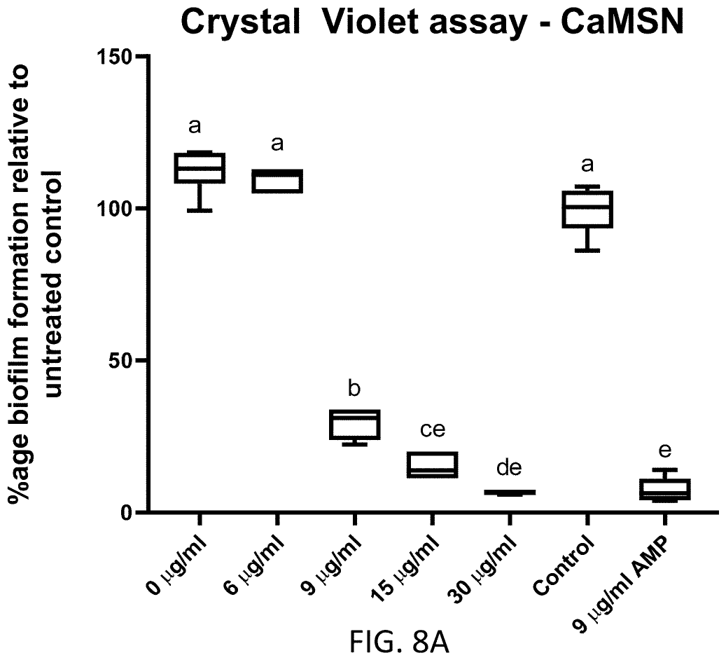

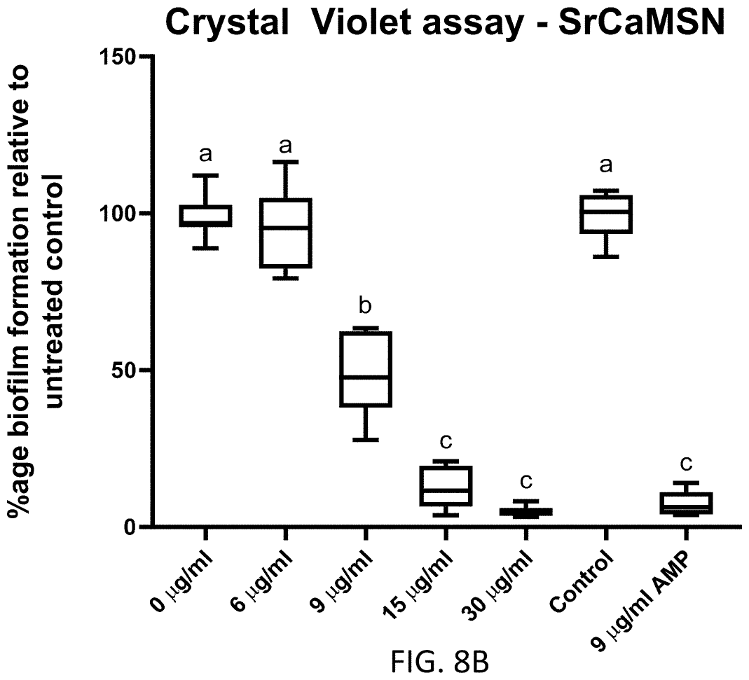

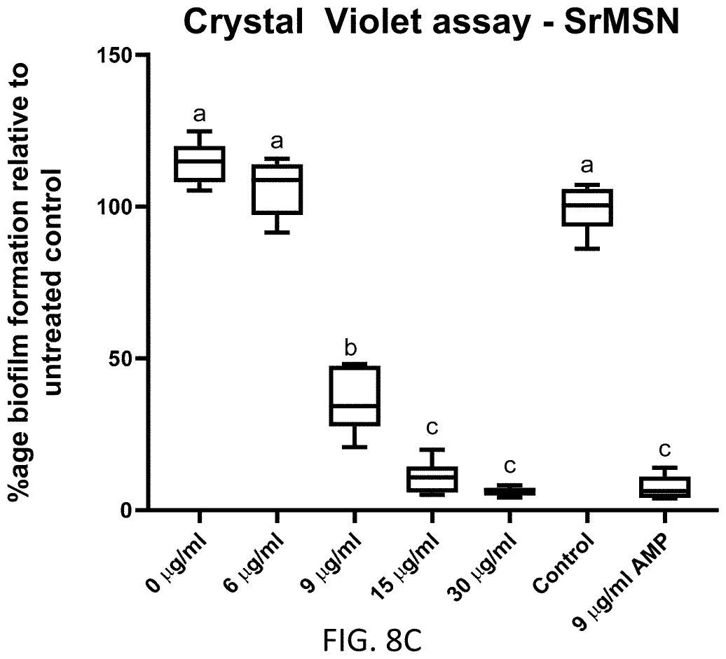

Overnight culture of S. aureus was treated the same was using method discussed for FIGS. 7A-7C, after which the biofilm mass formed in presence of different treatments was determined using crystal violet assay. Briefly, media was aspirated carefully with disturbing the formed biofilm and was washed with DI-water three times after which the biofilm was stained with 0.1% crystal violet stain for 15 minutes. Samples were washed again to remove unbound stain which was eluted in 30% acetic acid and absorbance measured at 550 nm using microplate reader (Synergy HT, Biotek). Significant differences in absorbance intensity (measure of total biofilm mass) was observed for bacteria treated with AMP alone or AMP-loaded MSNs relative to MSN alone and PBS control. The reduction in absorbance was observed in a dose dependent manner for cells treated with AMP-loaded CaMSN and SrMSNs at different tested concentrations. FIGS. 8A-8C show crystal violet biomass of USA300 cultured in presence of AMP-loaded MSNs, MSN alone and AMP added directly to the bacterial suspension. Values on x-axis correspond to the loaded AMP concentration. Statistical analysis: One-way ANOVA; post Bonferroni's test; letters with different symbol were significantly different (p<0.05).

Overnight culture of S. aureus was treated the same was using method discussed for FIGS. 7A-7C. A LIVE/DEAD assay was performed to visualize cell colonization of surfaces and membrane integrity of all experimental groups. After bacterial culture, samples were washed in PBS and stained on ice in SYTO 9 green stain and propidium iodide red stain in ultrapure water following the manufacturer's instructions (FilmTracer LIVE/DEAD, Thermo-Fisher, USA). Cells with an intact membrane fluoresce green, whereas cells with a compromised membrane, an indication of bacterial death, fluoresce red. The presence of AMP either alone or AMP-loaded MSNs resulted in significant membrane-compromised bacteria as indicated by significant amounts of stained cells, confirming antibacterial capability of AMP-loaded MSNs which was not evident in presence of MSNs without peptide.

Assessing the GelMA hydrogel mechano-physical properties as a surgical glue. GelMA hydrogels (6 mm diameter and 1 mm thickness) were fabricated at 12 wt % macromer concentration using 0.05 wt % LAP and cross-linked for 180 seconds. As-prepared and swollen hydrogels were characterized to determine the swelling ratio, soluble fraction, and compressive modulus. For measuring the adhesive strength of the hydrogel to porcine skin and titanium Grade 5 (Ti-6A1-4V alloy), 30 μl of macromer was added between the porcine skin/titanium and gelatin coated glass slide and cured for 90 seconds. The adhesive strength was measured at a speed of 1.5 mm/min until failure using a MTS Criterion 41 equipped with a 100 N load cell. Table 1 shows the results and indicates that the fabricated GelMA hydrogel was a cross-linked matrix with the ability to adhere to both metal and skin tissue.

TABLE 1
Summary of physico-mechanical properties
of fabricated GelMA hydrogel

| Property | Outcome |
|---|---|
| Soluble Fraction (%) | 9.9 ± 4.1 |
| Swelling ratio (q) | 6.6 ± 0.3 |
| Compressive Modulus (kPa) | 32.9 ± 6.8 |
| Adhesion to Grade 5 Titanium (kPa) | 49.2 ± 3.7 |
| Adhesion to Porcine Skin (kPa) | 17.7 ± 0.7 |

Assessing the ability of GelMA hydrogel to support cell attachment of keratinocytes and fibroblasts. GelMA hydrogels (6 mm diameter and 1 mm thickness) were fabricated at 12 wt % macromer concentration using 0.05 wt % LAP and cross-linked for 180 seconds. Cultured epithelial keratinocytes (HaCaTs, AddexBio) and human dermal fibroblasts (HDFs, ATCC) were passaged, counted and seeded on as-prepared hydrogels and cultured for 72 h. After 72 h, cells were fixed and stained with Integrin (34 (Igβ4, Novus Biologics; NB100-65599) and Integrin αvβ1 (Igαvβ1, Bioss Antibodies; BS-2016R-CY3) for HaCaTs and Integrin αvβ1 (Igαvβ1) for HDFs via immunofluorescence. Igβ4 is a key marker for hemidesmosome formation and Igαvβ1 plays an important role in cell migration and adhesion during re-epithelialization. The results indicated the ability of the GelMA hydrogel to support the growth of both HaCaTs and HDFs. The results also indicated that the hydrogels supported the expression of Igβ4 for HaCaTs and Igαvβ1 for both HaCaTs and HDFs. This analysis showed that the fabricated GelMA hydrogel support the expression of key markers associated with keratinocytes and fibroblast involved in cell attachment and re-epithelialization.

Assessing the ability of SrMSN-incorporated GelMA hydrogel to support cell attachment of keratinocytes and fibroblasts. GelMA hydrogels (6 mm diameter and 1 mm thickness) were fabricated at 12 wt % macromer concentration using 0.05 wt % LAP and cross-linked for 180 seconds. For SrMSN-incorporated hydrogels, SrMSNs were autoclaved and resuspended in phosphate buffer saline at a concentration of 30 mg/ml and then added to the GelMA macromer at a concentration of 1.5, 3 and 5 mg/ml and cross-linked. Cultured HaCaTs and HDFs were passaged, counted and seeded on as-prepared hydrogels and cultured for 72 h. After 72 h, cells were fixed and stained with Integrin β4 (Ig β4) and Integrin αvβ1 (Igαvβ1) for HaCaTs and Integrin αvβ1 (Igαvβ1) for HDFs via immunofluorescence. Igβ4 is a key marker for hemidesmosome formation and Igαvβ1 play an important role in cell migration and adhesion during re-epithelialization. This analysis showed the ability of the hydrogel GelMA control and GelMA+1.5 to 5 mg/ml SrMSN to support the growth of 1) HaCaTs expressing either Igβ4 or Igαvβ1 and 2) HDFs expressing Igαvβ31. The SrMSN-incorporated GelMA nanocomposite hydrogels support the expression of key markers necessary associated with keratinocytes and fibroblast involved in cell attachment and re-epithelialization and did not impair cell attachment and functionality.

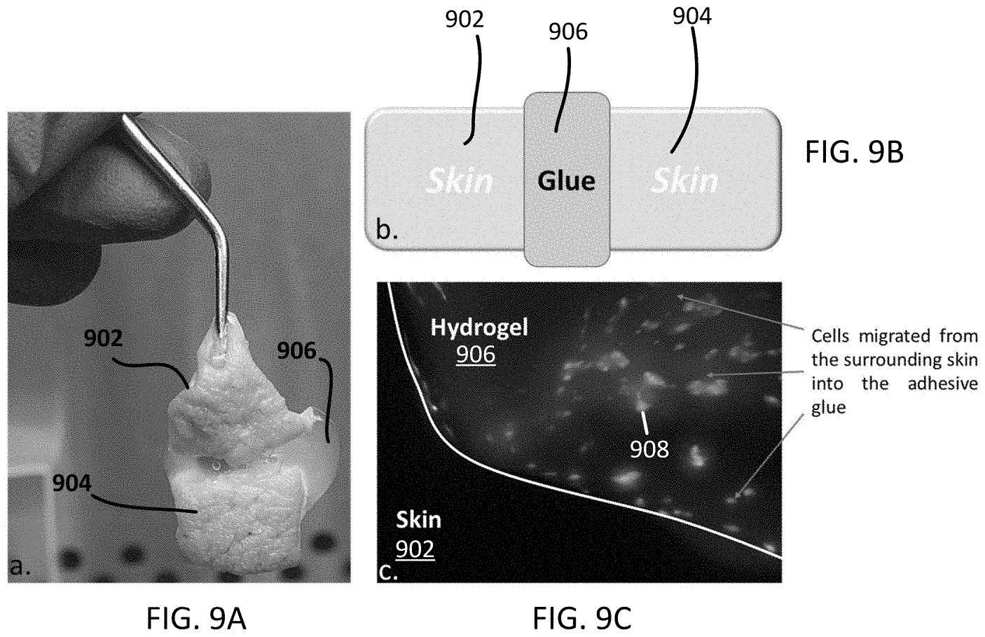

Assessing the ability of GelMA hydrogel to support skin tissue attachment and migration of cells from skin tissue into the hydrogel. Porcine skin tissue was obtained from visible heart labs and was cleaned to remove underlying fat tissue and was cut into small pieces using a sharp scalpel blade. Two pieces of porcine skin tissue were placed close to each other (2 mm gap between two pieces) and the macromer mixed with photoinitiator and was crosslinked at 400 nm. The skin tissue with hydrogel was placed in a six well plate and cultured for another 14 days in FBS (Gibco, USA, Fisher Scientific) supplemented with DMEM (Fisher Scientific). The results suggest the GelMA-based hydrogel acts as a glue that holds skin tissues together and cell migration from the skin into hydrogel was observed after 14 days of skin tissue cultured at liquid-air interface. FIG. 9A shows the ability of the glue to hold two pieces of human skin tissue 902, 904 together post cross-linking with the hydrogel 906. FIG. 9B shows a schematic of the bonding in FIG. 9A. FIG. 9C is an image showing migration of cells 908 from the skin tissue 902 into the adhesive 906 after 14 days of culture in media.

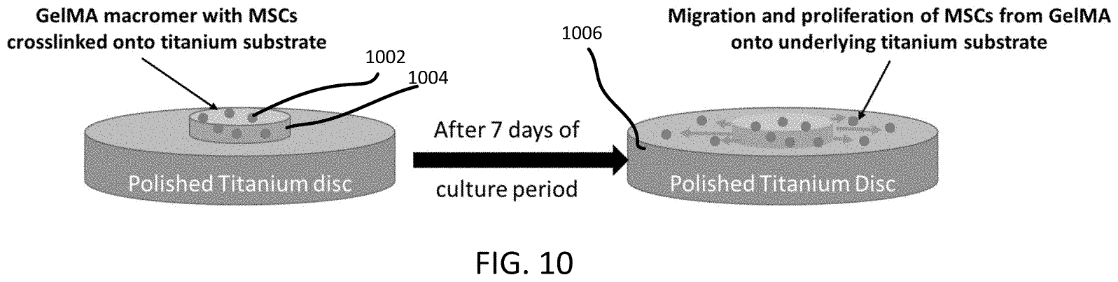

Assessing the ability of cell-encapsulated GelMA hydrogel to survive and migrate from hydrogel onto underlying titanium substrates. MSCs were encapsulated in different macromer concentrations (5 wt %, 7.5 wt % and 10 wt %) at a cell density of 5×10⁶ cells/ml and the hydrogels were crosslinked on titanium samples. The cell encapsulated hydrogel coated titanium samples were cultured for another seven days. Samples were then stained with Calcein/propidium iodide (live/dead stain) and samples were imaged using Leica DM6 B upright fluorescent microscope at ×10 (0.32 PH1 at 1296×966 pixels). The results suggest that the encapsulated cells were proliferative and migrated from hydrogel and grew on underlying titanium substrates. No significant differences were observed between different hydrogel formulations. FIG. 10 depicts the migration of encapsulated MSCs 1002 from GelMA hydrogels 1004 of varying stiffnesses onto an underlying titanium substrate 1006.

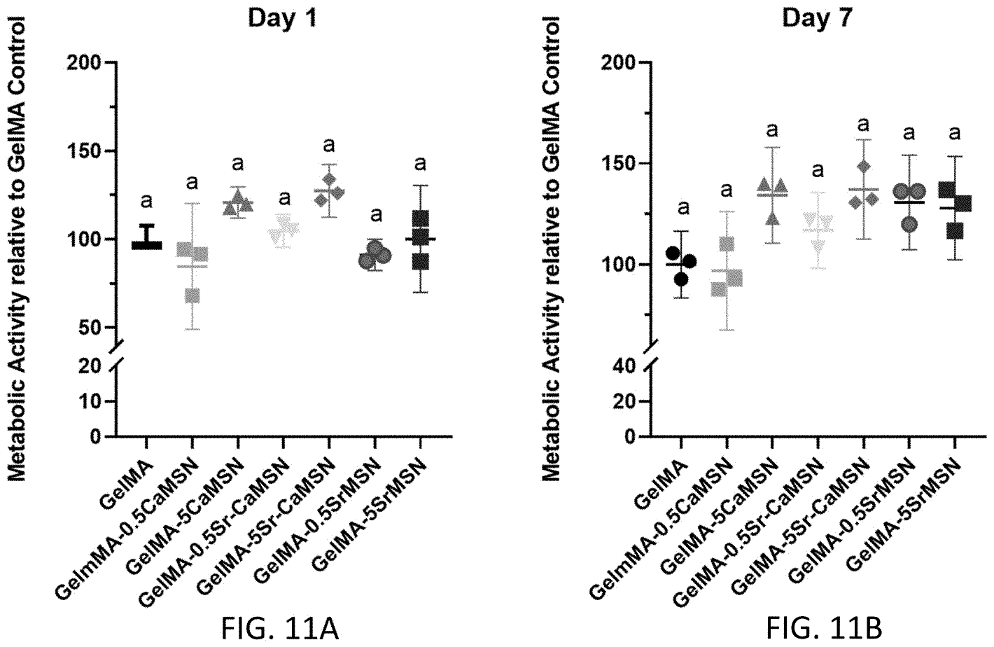

Assessing the viability of MSCs encapsulated within GelMA-MSN nanocomposite hydrogel. MSCs were encapsulated with 10 wt % GelMA hydrogel with MSNs (CaMSN, Sr-CaMSN and SrMSN at a concentration of 0.5 mg/ml and 5mg/ml at a cell density of 5x10⁶ cells/ml at LAP (photo-initiator concentration of 0.1% and cross-linking time of 30 seconds). Cell encapsulated hydrogels were cultured in FBS supplemented MEM medium (Stem Cell Technologies) for 7 days. Metabolic activity was assessed at day 1 and 7 using method discussed previously. The results suggest the different hydrogel formulations support stem cell growth with no significant differences observed between different hydrogel nanocomposites. FIGS. 11A and 11B show mesoporous MSNs-laden GelMA support cell viability (day 1) and proliferation (day 7), respectively, of encapsulated MSCs as determined by alamarBlue™. Statistical analysis: One-way ANOVA; post Bonferroni's test; letters with different symbol were significantly different (p<0.05).

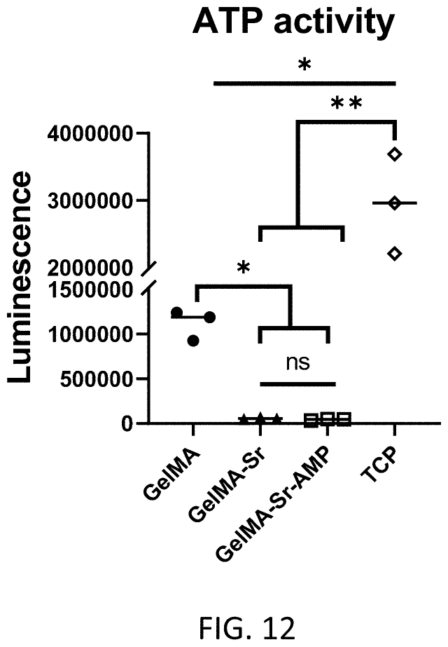

Assessing the antibacterial capability of GelMA hydrogel with SrMSN (with or without AMP) against USA300. This assessment is similar to that described with respect to FIGS. 7A-7C, except for the culture of bacterial suspension on different hydrogel formulations along with TCP control. Significant reduction in bacterial metabolic activity was observed when the cells were cultured on GelMA which was further reduced in presence of SrMSN with or without peptide relative to TCP control. FIG. 12 shows metabolic activity of USA300 cultured either on GelMA, GelMA with SrMSN and GelMA with AMP-loaded SrMSN. Statistical analysis: One-way ANOVA; post Bonferroni's test; letters with different symbol were significantly different (p<0.05).

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A bioactive adhesive composition comprising:
a hydrogel precursor; and
a multiplicity of metal-containing mesoporous silicate nanoparticles dispersed throughout the hydrogel precursor and comprising an antimicrobial peptide adsorbed on surfaces of the mesoporous silicate nanoparticles, incorporated in the mesoporous silicate nanoparticles, or both.

2. The composition of claim 1, wherein the hydrogel precursor comprises gelatin methacryloyl.

3. The composition of claim 1, wherein the metal-containing mesoporous silicate nanoparticles comprise calcium, strontium, or both.

4. The composition of claim 3, wherein the metal-containing mesoporous silicate nanoparticles comprise calcium or calcium and strontium, and a diameter of the metal-containing mesoporous silicate nanoparticles is in a range of about 150 nm to about 250 nm.

5. The composition of claim 3, wherein the metal-containing mesoporous silicate nanoparticles comprise strontium, and a diameter of the metal-containing mesoporous silicate nanoparticles is in a range of about 350 nm to about 450 nm.

6. The composition of claim 1, wherein the antimicrobial peptide has a loading efficiency of at least 50%.

7. The composition of claim 1, wherein the antimicrobial peptide comprises GL13K, 1018, DJK2, DJKS, hlf1-11, nisin, LL-37, or a combination thereof.

8. The composition of claim 1, wherein the metal-containing mesoporous silicate nanoparticles are configured to release the antimicrobial peptide over time.

9. The composition of claim 1, wherein the composition is photopolymerizable.

10. The composition of claim 9, wherein the composition is polymerizable under visible light.

11. The composition of claim 1, wherein the metal-containing mesoporous silicate nanoparticles comprise 0.5 wt % to 50 wt % of the composition.

12. The composition of claim 1, wherein the composition is configured to adhere to skin.

13. The composition of claim 1, wherein the composition is configured to adhere to metal.

14. The composition of claim 1, wherein the composition is configured to promote release of cytokines from soft tissue in contact with the composition.

15. A method of adhering skin to a metal surface, the method comprising:
disposing the adhesive composition of claim 1 on a metal surface;
contacting a portion of skin with the adhesive composition; and
curing the adhesive composition, thereby adhering the portion of the skin to the metal surface.

16. The method of claim 15, wherein curing the adhesive composition comprises irradiating the adhesive composition with visible light.

17. The method of claim 15, the metal surface is a surface of a percutaneous implant.

18. The method of claim 15, wherein the metal surface comprises titanium.

19. The method of claim 15, wherein adhering the portion of the skin to the metal surface forms a seal between the portion of the skin and the metal surface.

20. The method of claim 15, wherein curing the adhesive comprises converting the hydrogel precursor to a hydrogel.
